(19) United States
(12) Patent Application Publication
(10) Pub. No.: US 2004/0033226 A1
(43) Pub. Date: Feb. 19, 2004

(54) Osteopontin, oligodendrocytes and myelination

(75) Inventors: Jean Merrill, Whippany, NJ (US); Su-Fen Pu, Bridgewater, NJ (US); Olga Khorkova, North Haledon, NJ (US); Zhongqi Ji, Bridgewater, NJ (US); Fernando Camacho, Union, NJ (US); Steve Busch, Stewartsville, NJ (US); Min Wang, Blue Bell, PA (US); Ting Chen, Bridgewater, NJ (US); Robert Dinerstein, Flemington, NJ (US); Zhengbin Yao, Sugar Land, TX (US); Yangde Chen, Stewartsville, NJ (US); Xin Zhang, Piscataway, NJ (US); Karen Chandross, Somerset, NJ (US); Lan Lee, Pluckemin, NJ (US)

Correspondence Address: ROSS J. OEHLER, AVENTIS PHARMACEUTICALS INC., ROUTE 202-206, MAIL CODE: D303A, BRIDGEWATER, NJ, 08807, US

(73) Assignee: Aventis Pharmaceuticals Inc., Bridgewater, NJ

(21) Appl. No.: 10/602,927

(22) Filed: Jun. 24, 2003

Related U.S. Application Data
(60) Provisional application No. 60/391,035, filed on Jun. 25, 2002.

(30) Foreign Application Priority Data
Oct. 16, 2002 (GB) 0224017.4

(51) Int. Cl. A61K 39/395

(52) U.S. Cl. 424/143.1; 424/145.1

(57) ABSTRACT

Osteopontin is a marker for oligodendrocyte precursor cells. Osteopontin also modulates oligodendrocyte differentiation and central nervous system myelination. Osteopontin induces migration of oligodendrocyte precursors.

### PRIORITY CLAIM

This application claims priority under 35 U.S.C. 119 from U.S. Ser. No. 60/391,035 filed on Jun. 25, 2002 and British Application No. 0224017.4 filed on Oct. 16, 2002, wherein said applications are hereby incorporated by reference herein in their entireties.

### FIELD OF INVENTION

Osteopontin impacts oligodendrocyte development. Because oligodendrocytes are instrumental in remyelination, osteopontin has a role in remyelination.

### BACKGROUND

Osteopontin is a phosphoprotein with a molecular weight ranging from 44-66 kD, the variation depending on the species of origin and the degree of modification, such as glycosylation. Osteopontin is known also as secreted phosphoprotein 1, bone sialoprotein, urinary stone protein and early T lymphocyte activation-1 (eta-1) antigen. Osteopontin is acidic and binds calcium. Osteopontin contains the adhesive amino acid sequence, Arg-Gly-Asp (RGD) that interacts primarily with αᵥβ₃ integrin on the cell surface, but also with αᵥβ₁, αᵥβ₅, α₉β₁ and α₄β₁. Osteopontin is expressed in a number of different cell types and may serve a number of functions. For example, osteopontin may have a role in tissue matrix remodeling or bone resorption, as an attachment factor, in immune cell function and in metastasis. In human, the single gene encoding osteopontin is located on the long arm of chromosome 4.

Osteopontin has been found also in various cells of the nervous system. Primary sensory neurons in ganglia and in various parts of the brain have been found to express osteopontin. Thus, osteopontin may have a role in neural development and function.

Multiple sclerosis is characterized in part by demyelination of neural processes. Demyelination is observed in several other debilitating disorders of the central and peripheral nervous system including spinal cord injury, neural infections and neuropathies.

In diseases characterized by demyelination and in rodent models with demyelination, there is evidence that remyelination can occur. For example, in multiple sclerosis, there appears to be cycles of demyelination and remyelination.

### SUMMARY OF THE INVENTION

It is an object of the instant invention to provide a method wherein levels of osteopontin impact oligodendrocyte differentiation, numbers of oligodendrocytes and myelination. Controlling myelination is beneficial because demyelination of neurons can lead to improper signal conduction. In the central nervous system, myelin in formed by oligodendrocytes.

Another object of the instant invention is to provide a method for modulating myelination by modulating osteopontin levels.

An additional object of the instant invention is to provide a method for modulating oligodendrocyte differentiation by modulating osteopontin levels.

Yet another object of the instant invention is to modulate oligodendrocyte migration by modulating osteopontin levels. The modulation of osteopontin levels can occur by impacting osteopontin metabolism or receptor metabolism, such as of αᵥβ₃. The effects of osteopontin may be mediated or affected by T_{HI} cells.

Those and other objects of the invention were obtained in the observation that osteopontin prevents differentiation of oligodendrocyte precursor cells. Thus, a reduction in osteopontin levels results in accelerated differentiation of oligodendrocytes. That reduction in differentiation is accompanied by proliferation of precursor cells. The increased number of precursor cells results in a greater number of precursors and ultimately, of oligodendrocytes, for example, in the vicinity of demyelination. Osteopontin also stimulates migration of oligodendrocytes to areas of demyelination. The greater numbers of oligodendrocytes enhances remyelination. Those objects also can be achieved by impacting the ability of target cells to acquire, to bind and/or to respond to osteopontin. Cells that cannot acquire and react to osteopontin will not differentiate, instead proliferating.

### DETAILED DESCRIPTION OF THE INVENTION

It has been observed that osteopontin causes cells of a human oligodendrocyte line to present unusual growth characteristics. On exposure to osteopontin, cells of the oligodendrocyte cell line changed from a large cuboidal shape to a smaller bipolar shape resembling progenitor cells. Osteopontin did not have the same effect on neuroblastoma cells. The morphologic characterization of oligodendrocytes and precursors thereof is known and can be used to determine whether a molecule has an activity that mimics the dedifferentiating or differentiation inhibiting activity of osteopontin. Thus, osteopontin causes a dedifferentiation of oligodendrocytes and/or prevents differentiation of what appear to be precursors or less differentiated cells into mature oligodendrocytes.

As osteopontin levels wane, the oligodendrocyte progenitor cells begin to differentiate into mature oligodendrocytes that are able, among other functions, to myelinate neurons.

Osteopontin also serves as a chemotactic factor. Osteopontin is present in remyelinating lesions in the rat. Oligodendrocyte progenitors migrate into those lesions where those cells contribute to remyelination. The cells move in an osteopontin gradient moving from areas of lower concentration of osteopontin to areas of higher concentration of osteopontin. Osteopontin causes migration of responsive cells, including progenitor cells, into demyelinated areas where remyelination occurs.

Therefore, osteopontin suppresses differentiation of oligodendrocyte progenitors as well as induces such oligodendrocyte progenitors to migrate to areas containing greater concentrations of osteopontin.

Osteopontin also induces macrophages, microglia or both to migrate into demyelinated sites. There, those cells “clean up” myelin debris. The removal of myelin debris is a prerequisite for remyelination.

For the purposes of the instant invention, because of the reciprocal relationship between osteopontin and the receptor thereof, the artisan will recognize that for the purposes of obtaining the desired goals of chemoattraction and/or differentiation suppression, manipulation of either can be practiced. Thus, a teaching herein relating to either osteopontin or the receptor should be considered equivalent and applicable to either if the desired result is obtained, such as chemoattraction or differentiation control.

The gene sequence of osteopontin is known and available to the artisan. Thus, routine methods can be used to obtain expression of osteopontin in selected cells using known recombinant nucleic acid techniques (e.g., as described in Sambrook et al., eds., “Molecular Cloning: A Laboratory Manual”, 2^{nd} ed., Cold Spring Harbor Laboratory Press, Cold Spring Harbor, NY, 1989 and “Current Protocols in Molecular Biology”, John Wiley & Sons, N.Y. 1989). The osteopontin nucleic acid is subcloned in a suitable vector, introduced into a suitable cell to obtain expression. The vector either may be maintained extrachromosomally, or can be configured to integrate into the host genome. Various control elements can be incorporated into the vector to obtain high level expression of osteopontin or controlled expression therefore. For example, enhancers, particular promoters, splice sites and the like can be used.

In certain embodiments, the recombinant mammalian expression vector is capable of directing expression of the nucleic acid preferentially in a particular cell type (e.g., tissue-specific regulatory elements can be used to express the nucleic acid). Tissue-specific regulatory elements are known in the art. Non-limiting examples of suitable tissue-specific promoters include, for example, neuron-specific promoters (e.g., the neurofilament promoter; Byrne et al., Proc. Natl. Acad. Sci. USA (1989) 86:5473-5477) and promoters that are specific for fibroblasts, endodermal cells, microglial cells and oligodendrocytes. Developmentally-regulated promoters also are encompassed, for example the murine hox promoters (Kessel et al., Science (1990) 249:374-379) and the α-fetoprotein promoter (Campes et al., Genes Dev. (1989) 3:537-546).

Alternatively, control of expression can be obtained using particular inducible promoters, as known in the art. For example, the tetracycline/tet activator system employing operator elements that bind and are induced by an activator protein can be used. However, the activator binds tetracycline which reaction prevents binding of the activator to the operator elements. Hence, tetracycline inhibits expression of a transgene cloned downstream of the operator elements.

Because a goal of the instant invention is to obtain osteopontin function, it is possible to obtain truncated or modified forms of osteopontin for enhanced or reduced expression, ease of manipulation or to minimize unwanted effects. Thus, the osteopontin nucleic acid or protein can be manipulated by known techniques to obtain truncated or modified versions of osteopontin that bind to the osteopontin receptor and retain the ability to yield or solicit the particularized effects desired in the instant invention.

Mutations in the nucleic acid can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in that the amino acid residue is replaced with an amino acid residue having a similar side chain. For example, families of amino acid residues having similar side chains are defined in the art. The families include amino acids with basic side chains (e.g., lysine, arginine and histidine), acidic side chains (e.g., aspartic acid and glutamic acid), uncharged polar side chains (e.g., glycine, asparagine, glutamine, serine, threonine, tyrosine and cysteine), nonpolar side chains (e.g., alanine, valine, leucine, isoleucine, proline, phenylalanine, methionine and tryptophan), branched side chains (e.g., threonine, valine and isoleucine) and aromatic side chains (e.g., tyrosine, phenylalanine, tryptophan and histidine). Thus, a predicted nonessential amino acid residue in osteopontin or the receptor thereof preferably can be replaced with another amino acid residue from the same side chain family. Alternatively, mutations can be introduced randomly along all or part of the osteopontin coding sequence, such as by saturation mutagenesis, and the resultant mutants can be screened for osteopontin activity to identify mutants that retain activity. Following mutagenesis, the encoded protein can be expressed recombinantly and the activity of the protein can be determined.

The instant invention also encompasses antisense nucleic acid molecules, i.e., molecules that are complementary to a sense nucleic acid encoding a protein, e.g., complementary to the coding strand of a double-stranded cDNA molecule or complementary to an mRNA sequence, essentially to reduce expression of osteopontin or the receptor thereof. Accordingly, an antisense nucleic acid can hydrogen bond to a sense nucleic acid. The antisense nucleic acid can be complementary to an entire osteopontin or receptor coding strand or to only a portion thereof, e.g., all or part of the protein coding region (or open reading frame). An antisense nucleic acid molecule can be antisense to a noncoding region of the coding strand of a nucleotide sequence encoding osteopontin or the receptor thereof. The noncoding regions (“5′ and 3′ untranslated regions”) are the 5′ and 3′ sequences that flank the coding region and are not translated into amino acids but may have a regulatory role. The flanking site may be targeted if normal expression of osteopontin or receptor thereof is reliant thereon.

Given the coding strand sequences encoding osteopontin or the receptor thereof are known, antisense nucleic acids of the invention can be designed according to the rules of Watson & Crick base pairing. The antisense nucleic acid molecule can be complementary to the entire coding region of the appropriate mRNA, but more preferably is an oligonucleotide that is antisense to only a portion of the coding or noncoding region of the relevant MRNA. For example, the antisense oligonucleotide can be complementary to the region surrounding the translation start site of the osteopontin mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be synthesized chemically using naturally occurring nucleotides or variously modified nucleotides designed to increase the biological stability of the molecules or to increase the physical stability of the duplex formed between the antisense and sense nucleic acids, e.g., phosphorothioate derivatives, phosphonate derivatives and acridine-substituted nucleotides can be used.

Such an antisense technology can be used to produce a knockout animal wherein the osteopontin gene or osteopontin receptor gene is rendered non-functional. The antisense molecule can be directed to a coding sequence or to a non-coding sequence, such as a regulatory sequence, such as a promoter. The antisense molecule can be RNA or DNA. Generally the inactivating effect is transient as the antisense molecule is degraded or diluted by cell division. However, the antisense construct can be configured with constitutive or inducible promoters to obtain more tonic expression of the antisense molecule.

A more permanent inactivation can be obtained by having the antisense construct integrate into the genome of the host cell. That can be accomplished, for example, by homologous recombination, as known in the art. Homologous recombination in germ cells or precursor cells can yield an animal where the osteopontin gene or osteopontin receptor gene is inactive in a tissue, organ or the entire organism, the last scenario resulting in a knockout transgenic organism

Examples of modified nucleotides that can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, β-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-adenine, 7-methylguanine, 5-methylaminomethyluracil, 5-methoxyaminomethyl-2-thiouracil, β-D-mannosylqueosine, 5-methoxycarboxymethyluracil, 5-methoxyuracil, 2-methylthio-N⁶-isopentenyladenine, uracil-5-oxyacetic acid, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl)uracil and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules of the invention typically are administered to a subject or generated in situ so as to hybridize with or bind to cellular mRNA and/or genomic DNA encoding osteopontin or the receptor thereof to inhibit expression of the protein, e.g., by inhibiting transcription and/or translation. The hybridization can be by conventional nucleotide complementation to form a stable duplex, or, for example, in the case of an antisense nucleic acid molecule that binds to DNA duplexes, through specific interactions in the major groove of the double helix, or to a regulatory region of osteopontin or the receptor thereof.

An example of a route of administration of antisense nucleic acid molecules of the invention is direct injection at a tissue site. Alternatively, antisense nucleic acid molecules can be modified to target selected cells and then administered systemically. For example, for systemic administration, antisense molecules can be modified such that the molecules specifically bind to receptors or antigens expressed on a selected cell surface, e.g., by linking the antisense nucleic acid molecules to peptides or antibodies that bind to cell surface receptors or antigens. The antisense nucleic acid molecules also can be delivered to cells using the vectors described herein. To achieve sufficient intracellular concentrations of the antisense molecules, the antisense nucleic acid molecule can be placed under the control of a strong promoter, a pol II or pol III promoter is preferred.

An antisense nucleic acid molecule of the invention can be an α-anomeric nucleic acid molecule. An α-anomeric nucleic acid molecule forms specific double-stranded hybrids with complementary RNA in that the strands run parallel to each other (Gaultier et al., Nucleic Acids Res. (1987)15:6625-6641). The antisense nucleic acid molecule also can comprise a methylribonucleotide (Inoue et al., Nucleic Acids Res. (1987) 15:6131-6148) or a chimeric RNA-DNA analogue (Inoue et al., FEBS Lett. (1987) 215:327-330).

The invention also encompasses ribozymes. Ribozymes are catalytic RNA molecules with ribonuclease activity that are capable of cleaving a single-stranded nucleic acid, such as an mRNA, to that the ribozyme is hybridized. Thus, ribozymes (e.g., hammerhead ribozymes (described in Haselhoff et al., Nature (1988) 334:585-591)) can be used to cleave catalytically the relevant MRNA transcripts to inhibit translation of that mRNA. A ribozyme having specificity for an osteopontin or receptor nucleic acid can be designed based on the nucleotide sequence of osteopontin or receptor. For example, a derivative of a Tetrahymena L-19 IVS RNA can be constructed in which the nucleotide sequence of the active site is complementary to the nucleotide sequence to be cleaved in the osteopontin or receptor mRNA, see, e.g., U.S. Pat. Nos. 4,987,071 and 5,116,742. Alternatively, osteopontin mRNA can be used to select a catalytic RNA having a specific ribonuclease activity from a pool of RNA molecules, see, e.g., Bartel et al., Science (1993) 261:1411-1418.

The invention also encompasses nucleic acid molecules that form triple helical structures. For example, osteopontin gene expression can be inhibited by targeting nucleotide sequences complementary to the regulatory region of osteopontin (e.g., the osteopontin promoter and/or enhancers) to form triple helical structures that prevent transcription of the osteopontin gene in target cells, see generally, Helene, Anticancer Drug Dis. (1991) 6(6):569; Helene Ann, NY Acad. Sci. (1992) 660:27; and Maher, Bioassays (1992) 14(12):807.

In preferred embodiments, the nucleic acid molecules of the invention can be modified at the base moiety, sugar moiety or phosphate backbone to improve, e.g., the stability, hybridization or solubility of the molecule. For example, the deoxyribose phosphate backbone of the nucleic acids can be modified to generate peptide nucleic acids (see Hyrup et al., Bioorganic & Medicinal Chemistry (1996) 4:5). As used herein, the terms “peptide nucleic acids” or “PNAs” refer to nucleic acid mimics, e.g., DNA mimics, in that the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) supra; Perry-O'Keefe et al., Proc. Natl. Acad. Sci. USA (1996) 93:14670.

As disclosed herein base pairing is not essential to block osteopontin expression or receptor expression, aptamers are nucleic acids that interact with a particular nucleic acid much in the same way proteins interact with other proteins or with nucleic acids, that is depending on change, other attractive forces and conformation.

Moreover certain mimetics need not be nucleic acid, a protein or carbohydrate can have specificity for an osteopontin or receptor nucleic acid thereby preventing transcription or translation thereof.

When the osteopontin or biologically active portion thereof is produced recombinantly, it also is preferably substantially free of culture medium, i.e., culture medium represents less than about 20%, 10% or 5% or less of the volume of the protein preparation.

When osteopontin or a portion thereof is produced by chemical synthesis, it is preferably substantially free of chemical precursors or other chemicals, i.e., it is separated from chemical precursors or other chemicals that are involved in the synthesis of the protein. Accordingly such preparations of osteopontin have less than about 30%, 20%, 10% or 5% or less (by dry weight) of chemical precursors or non-osteopontin chemicals.

Because oligodendrocyte cells and the precursors thereof are important in remyelination, a suitable host cell would be an oligodendrocyte or an oligodendrocyte precursor. Expression of osteopontin by such a transformed cell can serve as a focal point for migration of naturally occurring oligodendrocyte precursors or oligodendrocytes as the site of the transformed cells.

Suitable host cells for transformation are glial cells such as microglial cells that secrete osteopontin. Again, the osteopontin produced by the transformed glial cells will provide a stimulus for migration of oligodendrocyte precursors to a particular site. The osteopontin also will yield delayed differentiation of oligodendrocytes. The reduction of oligodendrocyte maturation and proliferation of oligodendrocyte precursor cells increases the eventual number of oligodendrocytes at a particular site thereby enhancing or increasing the remyelination process.

Another suitable cell for transformation is a fibroblast or other endodermal cell that can be localized to a particular site. Fibroblasts can express osteopontin at a particular site thereby increasing the number of oligodendrocyte precursors.

Such transformed cells can be placed at a site requiring remyelination, often in the central nervous system. Thus, various known surgical methods for implanting transformed cells can be used to obtain cells expressing osteopontin at the desired site.

Alternatively, purified osteopontin or a truncated, biologically effective portion thereof can be instilled at a site or can be placed in a release device, such as a silastic implant and the like. Thus, osteopontin is released locally at a predetermined rate at a site of desired remyelination. The osteopontin can serve to attract cells to that site.

Being expressed by oligodendrocyte precursor cells and microglia, for example, osteopontin is a marker for those populations of cells. Thus, detection of osteopontin is a means for identifying, for example, oligodendrocyte precursor cells. A number of different methods can be used to exploit that property of the oligodendrocyte precursor cells, such as RIA, ELISA, TaqMan, Northern blot or a fluorescence assay.

For example, an antibody directed to osteopontin can be used to identify cells actively producing and secreting osteopontin. The antibody can be to osteopontin or to the osteopontin MRNA, either alone or when bound with ribosomes. As known in the art, the antibody can be labeled directly or an indirect label can be used wherein the osteopontin antibody is identified by a secondary molecule, such as a binding molecule or another antibody.

A number of different molecules can be used to label the osteopontin antibody directly, such as a radioactive label, an enzymic label, a fluorescent compound label, a chemiluminescent compound label and so on. The various labels can be attached to the antibody molecule using techniques known in the art, such as using a labeled amino acid precursor, by some other metabolic means, for example, by some chemical means resulting in a covalent binding of the label to the antibody molecule, using a linker molecule and so on. Then a suitable detection means is used to ascertain for the presence of the labeled antibody molecule. Examples of detectable substances include various enzymes, prosthetic groups, fluorescent materials, luminescent materials, bioluminescent materials and radioactive materials. Examples of suitable enzymes include horseradish peroxidase, alkaline phosphatase, galactosidase or acetylcholinesterase. Examples of suitable prosthetic group complexes include streptavidin/biotin and avidin/biotin. Examples of suitable fluorescent materials include umbelliferone, fluorescein, fluorescein isothiocyanate, rhodamine, dichlorotriazinylamine fluorescein, dansyl chloride or phycoerythrins. An example of a luminescent material is luminol. Examples of bioluminescent materials include luciferase, luciferin and aequorin. Examples of suitable radioactive materials include ¹²⁵I, ¹³¹I, ³⁵S or ³H.

In a case where detection of bound antibody is indirect, an antibody that binds to the osteopontin antibody can be used. For example, if the osteopontin antibody is made in a non-human species, the second antibody can be an antibody directed to a species allotypic determinant. Alternatively, an idiotypic determining of the osteopontin antibody can be the target. Alternatively, other binding molecules that will enable identifying the osteopontin antibody can be used. For example, if the osteopontin antibody is an IgG molecule recognizable by protein A or protein G, the protein A or protein G can be the labeled reporter molecule. The various labels discussed hereinabove can be used to label the protein A or protein G. Another such binding pair of molecules is the use of biotin to label the antibody molecule and then using a labeled avidin molecule to locate the biotinylated antibody molecule.

Alternatively, nucleic acids can be used to detect the osteopontin present in oligodendrocyte precursor cells. Such nucleic acids are known as aptamers and can be obtained from oligonucleotide libraries as known in the art.

Osteopontin nucleic acids also can be detected. In that case, a suitable means to detect expression of osteopontin would be to monitor presence of osteopontin message. Known methods for detecting message include Northern Blot, Taqman® analysis, in situ hybridization and so on. The osteopontin-specific nucleic acid probe is labeled and allowed to hybridize with osteopontin MRNA. Following hybridization, the osteopontin probe that can be labeled by any of a variety of means as discussed hereinabove, is revealed using the appropriate detection means. As with the use of a protein-based system discussed hereinabove, the osteopontin probe nucleic acid either can be labeled directly or another labeled reporter molecule can be used to identify bound probe.

As a chemoattractant molecule for oligodendrocyte precursor cells, osteopontin can be used to characterize and isolate oligodendrocyte precursor cells and motile oligodendrocytes. Assays to monitor cell motility are known in the art, for example, osteopontin can be entrapped within a semisolid matrix such as agarose, and then cells that are able to recognize and move toward the chemoattractant can be exposed to the bound osteopontin to determine if any cells can move into and in the matrix.

By capitalizing on the mobility of oligodendrocyte precursors and oligodendrocytes in response to osteopontin, as well as using a binding molecule such as an antibody molecule discussed hereinabove, a means for purifying oligodendrocyte precursor cells or oligodendrocytes can be configured. For example, cells that are attracted to osteopontin can be distinguished from a mixed population of cells. Also, for example, an antibody to osteopontin can be used to parse oligodendrocyte precursor cells or oligodendrocytes from a population of cells. For example, osteopontin antibody can be immobilized on a solid matrix, such as a bead or a culture plate surface and exposed to a mixed population of cells. Those cells that are bound by the antibody can be separated from those that are not bound by the antibody to obtain an enriched population of oligodendrocytes and/or oligodendrocyte precursor cells.

Alternatively, using a screening assay, for example, as taught herein, an agonist molecule that on binding to target cells induces migration of those cells to a desired site, can be identified and used. The agonist can be identified from a plurality of candidate compounds.

An “agonist” as used herein means moieties that activate the desired intracellular and/or cellular response(s) when bound to the osteopontin receptor.

A “partial agonist” as used herein means moieties (e.g., but not limited to osteopontin) that activate the intracellular and/or cellular response(s) when bound to the receptor to a lesser degree/extent then do agonists.

The term “candidate compound” herein means a moiety that is amenable to a screening technique. Such compounds can be identified by traditional drug discovery processes involving identification of an endogenous ligand specific for a receptor, and/or screening of candidate compounds against a receptor wherein such a screening requires a competitive assay to assess efficacy, or using biotechnical methods including use of antibodies and recombinant nucleic acids.

The invention provides a method (also referred to herein as a “screening assay”) for identifying modulators, i.e., candidate or test compounds or agents (e.g., peptides, peptidomimetics, small molecules or other drugs) that bind to the osteopontin receptor or have a stimulatory or inhibitory effect on osteopontin activity.

In one embodiment, the invention provides assays for screening candidate or test compounds that bind to or modulate the activity of the osteopontin receptor or biologically active portion thereof. The test compounds of the instant invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des (1997) 12:145).

Examples of methods for the synthesis of molecular libraries can be found in the art, for example in: DeWitt et al., Proc. Natl. Acad. Sci. USA (1993) 90:6909; Erb et al., Proc. Natl. Acad. Sci. USA (1994) 91:11422; Zuckermann et al., J. Med. Chem. (1994) 37:2678; Cho et al., Science (1993) 261:1303; Carrell et al., Angew Chem. Int. Ed. Engl. (1994) 33:2059; Carell et al., Angew Chem. Int. Ed. Engl. (1994) 33:2061; and Gallop et al., J. Med. Chem. (1994) 37:1233.

Libraries of compounds may be presented in solution (e.g., Houghten Bio/Techniques (1992) 13:412-421) or on beads (Lam, Nature (1991) 354:82-84), chips (Fodor, Nature (1993) 364:555-556), bacteria (U.S. Pat. No. 5,223,409), spores (U.S. Pat. Nos. 5,571,698; 5,403,484; and 5,223,409), plasmids (Cull et al., Proc. Natl. Acad. Sci. USA (1992) 89:1865-1869) or phage (Scott et al., Science (1990) 249:386-390; Devlin, Science (1990) 249:404-406; Cwirla et al., Proc. Natl. Acad. Sci. USA (1990) 87:6378-6382; and Felici, J. Mol. Biol. (1991) 222:301-310).

In one embodiment, an assay is a cell-based assay in which a cell that expresses an osteopontin receptor or a biologically active portion thereof, on the cell surface is contacted with a test compound and the ability of the test compound to bind to the osteopontin receptor is determined. The cell, for example, can be a yeast cell or a cell of mammalian origin. Determining the ability of the test compound to bind to the osteopontin receptor can be accomplished, for example, by coupling the test compound with a radioisotope or enzymatic label such that binding of the test compound to the osteopontin receptor or biologically active portion thereof can be determined by detecting the labeled compound in a complex. For example, test compounds can be labeled with ¹²⁵I, ³⁵S, ¹⁴C or ³H, either directly or indirectly and the radioisotope detected by direct counting of radioemmission or by scintillation counting. Alternatively, test compounds can be labeled enzymatically with, for example, horseradish peroxidase, alkaline phosphatase or luciferase and the enzymatic label detected by determination of conversion of an appropriate substrate to product.

The assay also can be competitive using labeled osteopontin. Thus, the assay comprises contacting a cell that expresses the osteopontin receptor or a biologically active portion thereof, on the cell surface with osteopontin to form an assay mixture, contacting the assay mixture with a test compound and determining the ability of the test compound to interact with the osteopontin receptor, wherein determining the ability of the test compound to interact with the osteopontin receptor comprises determining the ability of the test compound to bind preferentially to the osteopontin receptor a biologically active portion thereof as compared to the known compound.

In another embodiment, an assay is a cell-based assay comprising contacting a cell expressing the osteopontin receptor, or a biologically active portion thereof, on the cell surface with a test compound and determining the ability of the test compound to modulate (e.g., stimulate or inhibit) the activity of the osteopontin receptor or biologically active portion thereof. Determining the ability of the test compound to modulate the activity of the osteopontin receptor or a biologically active portion thereof can be accomplished, for example, by determining the ability of the osteopontin receptor to bind to or to interact with osteopontin or in the production of IL12. A number of cell surface molecules are known to bind osteopontin, such as CD44, and the αᵥ, α₆, β¹, β₃ and β₅ chains. The α and β chains are the subunits of the integrins.

Receptors can be activated by non-ligand molecules that necessarily do not inhibit ligand binding but cause structural changes in the receptor to enable, for example, receptor aggregation, dimerization or clustering that can cause activation.

Thus, antibodies can be raised to the various portions of the osteopontin receptor. Those antibodies that activate are determined by standard assays, such as monitoring IL12 production, and can be selected.

The antibodies can be made using known techniques. Because molecular mapping and particularly epitope mapping is involved, monoclonal antibodies probably are preferred. The monoclonal antibodies can be raised both to intact receptor expressed at the cell surface and peptides known to form at the cell surface. The method of Geysen et al., U.S. Pat. No. 5,998,577, can be practiced to obtain a plurality of relevant peptides.

Osteopontin receptor or a portion or fragment thereof, can be used as an immunogen to generate antibodies that bind same using standard techniques for polyclonal and monoclonal antibody preparation. The full-length osteopontin receptor can be used or, alternatively, the invention provides antigenic peptide fragments of osteopontin for use as immunogens. An antigenic peptide of osteopontin receptor comprises at least 8 (preferably 10, 15, 20, 30 or more) amino acid residues of the known sequence and encompasses an epitope of osteopontin receptor such that an antibody raised against the peptide forms a specific immune complex with same.

In a related aspect, epitopes encompassed by the antigenic peptide are regions of the osteopontin receptor that bind to specific portions of the receptor or are known to have a particular function.

An osteopontin receptor immunogen typically is used to prepare antibodies by immunizing a suitable subject, (e.g., rabbit, goat, mouse or other mammal) with the immunogen. An appropriate immunogenic preparation can contain, for example, recombinantly expressed osteopontin receptor or a chemically synthesized osteopontin receptor. The preparation further can include an adjuvant, such as Freund's complete or incomplete adjuvant, or similar immunostimulatory agent. Immunization of a suitable subject with an immunogenic osteopontin receptor preparation induces a polyclonal response.

The term “antibody” as used herein refers to immunoglobulin molecules and immunologically active portions of immunoglobulin molecules, i.e., molecules that contain an antigen-binding site that specifically binds to osteopontin receptor. A molecule that specifically binds to osteopontin receptor is a molecule that binds osteopontin receptor, but does not substantially bind other molecules in a sample, e.g., a biological sample that naturally contains osteopontin receptor. Examples of immunologically active portions of immunoglobulin molecules include F_{(ab)} and F_{(ab′)2} fragments that can be generated by treating the antibody with an enzyme such as pepsin.

The term “monoclonal antibody” or “monoclonal antibody composition”, as used herein, refers to a population of antibody molecules that contain only one species of an antigen-binding site capable of immunoreacting with a particular epitope of the osteopontin receptor. A monoclonal antibody composition thus typically displays a single binding affinity for a particular epitope.

The antibody titer in the immunized subject can be monitored over time by standard techniques, such as with an enzyme-linked immunosorbent assay (ELISA) using immobilized osteopontin receptor. If desired, the antibody molecules directed against osteopontin receptor can be isolated from the mammal (e.g., from the blood) and further purified by well-known techniques, such as protein A chromatography, to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler et al., Nature (1975) 256:495-497, the human B cell hybridoma technique (Kohler et al., Immunol. Today (1983) 4:72), the EBV hybridoma technique (Cole et al., Monoclonal Antibodies and Cancer Therapy, (1985), Alan R. Liss, Inc., pp. 77-96) or trioma techniques.

The technology for producing hybridomas is well known (see generally Current Protocols in Immunology (1994) Coligan et al., eds., John Wiley & Sons, Inc., New York, N.Y.). Briefly, an immortal cell line (typically a myeloma) is fused to lymphocytes (typically splenocytes) from a mammal immunized with an immunogen of interest as described above and the culture supernatants of the resulting hybridoma cells are screened to identify a hybridoma producing a monoclonal antibody that binds the osteopontin receptor.

Any of the many well known protocols used for fusing lymphocytes and immortalized cell lines can be applied for the purpose of generating monoclonal antibody (see, e.g., Current Protocols in Immunology, supra; Galfre et al., Nature (1977) 266:550-552; Kenneth, in Monoclonal Antibodies: A New Dimension In Biological Analyses, Plenum Publishing Corp., New York, N.Y. (1980); and Lerner, Yale J. Biol. Med. (1981) 54:387-402). Moreover, the ordinarily skilled worker will appreciate that there are many variations of such methods that also would be useful.

Typically, the immortal cell line (e.g., a myeloma cell line) is derived from the same mammalian species as the lymphocytes. For example, murine hybridomas can be made by fusing lymphocytes from a mouse immunized with an immunogenic preparation of the instant invention with an immortalized mouse cell line, e.g., a myeloma cell line that is sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. The myeloma lines are available from ATCC.

Typically, HAT-sensitive mouse myeloma cells are fused to mouse splenocytes using polyethylene glycol (“PEG”). Hybridoma cells resulting from the fusion then are selected using HAT medium that kills unfused and unproductively fused myeloma cells (unfused splenocytes die after several days because they are not transformed). Hybridoma cells producing a monoclonal antibody of the invention are detected by screening the hybridoma culture supernatants for antibodies that bind osteopontin, e.g., using a standard ELISA assay.

Preferably, the monoclonal antibodies are essentially or completely of human origin. Thus, chimeric antibodies having non-human complement determining regions in a human antibody framework can be used, practicing methods known in the art, PCT Publication No. WO 87/02671; Europe Patent Application No. 184,187; Europe Patent Application No. 171,496; Europe Patent Application No. 173,494; PCT Publication No. WO 86/01533; U.S. Pat. No. 4,816,567; Europe Patent Application No. 125,023; Better et al., Science (1988) 240:1041-1043; Liu et al., Proc. Natl. Acad. Sci. USA (1987) 84:3439-3443; Lin et al., J. Immunol. (1987) 139:3521-3526; Sun et al., Proc. Natl. Acad. Sci. USA (1987) 84:214-218; Nishimura et al., Canc. Res. (1987) 47:999-1005; Wood et al., Nature (1985) 314:446-449; Shaw et al., J. Natl. Cancer Inst. (1988) 80:1553-1559; Morrison, Science (1985) 229:1202-1207; Oi et al., Bio/Techniques (1986) 4:214; U.S. Pat. No. 5,225,539; Jones et al., Nature (1986) 321:552-525; Verhoeyan et al., Science (1988) 239:1534; and Beidler et al., J. Immunol. (1988) 141:4053-4060.

Human antibodies can be made using in vitro techniques. Completely human antibodies are particularly desirable for therapeutic treatment of human patients. Such antibodies can be produced using transgenic mice that are incapable of expressing endogenous immunoglobulin heavy and light chains genes, but that can express human heavy and light chain genes. Abgenix (Fremont, Calif.) makes human monoclonal antibodies in mice using mice carrying partial human immunoglobulin repertoires. The transgenic mice are immunized in normal fashion with a selected antigen. Monoclonal antibodies directed against the antigen can be obtained using conventional hybridoma technology. The human immunoglobulin transgenes harbored by the transgenic mice rearrange during B cell differentiation and subsequently undergo class switching and somatic mutation.

Alternative to preparing monoclonal antibody-secreting hybridomas, a monoclonal antibody can be identified and isolated by screening a recombinant combinatorial immunoglobulin library (e.g., an antibody phage display library) with osteopontin or the receptor thereof thereby to isolate immunoglobulin library members that bind osteopontin or the receptor thereof. Kits for generating and screening phage display libraries are commercially available (e.g., the Pharmacia Recombinant Phage Antibody System, Catalog No. 27-9400-01; and the Stratagene SurfZAP®Phage Display Kit, Catalog No. 240612).

Additionally, examples of methods and reagents particularly amenable for use in generating and screening antibody display libraries can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology (1991) 9:1370-1372; Hay et al., Hum. Antibody Hybridomas (1992) 3:81-85; Huse et al., Science (1989) 246:1275-1281; and Griffiths et al., EMBO J. (1993) 25(12):725-734.

Thus, using such an epitope, an antibody that binds thereto is identified. The heavy chain and the light chain of the antibody are cloned and used to create phage display Fab fragments. For example, the heavy chain gene can be cloned into a plasmid vector so that the heavy chain can be secreted from bacteria. The light chain gene can be cloned into a phage coat protein gene so that the light chain can be expressed on the surface of phage. A repertoire (random collection) of human light chains fused to phage is used to infect the bacteria that express the non-human heavy chain. The resulting progeny phage display hybrid antibodies (human light chain/non-human heavy chain). The selected antigen is used in a panning screen to select phage that bind the selected antigen. Several rounds of selection may be required to identify such phage.

Human light chain genes are isolated from the selected phage that bind the selected antigen. The selected human light chain genes then are used to guide the selection of human heavy chain genes as follows. The selected human light chain genes are inserted into vectors for expression by bacteria. Bacteria expressing the selected human light chains are infected with a repertoire of human heavy chains fused to phage. The resulting progeny phage display human antibodies (human light chain/human heavy chain).

Next, the selected antigen is used in a panning screen to select phage that bind the selected antigen. The selected phage display a completely human antibody that recognizes the same epitope recognized by the original selected, non-human monoclonal antibody. The genes encoding both the heavy and light chains are isolated and can be manipulated further for production of human antibody. The technology is described by Jespers et al. (Bio/Technology (1994) 12:899-903).

An osteopontin antibody (e.g., monoclonal antibody) can be used to isolate osteopontin by standard techniques, such as affinity chromatography or immunoprecipitation. Such an antibody can facilitate the purification of natural osteopontin from cells and of recombinantly produced osteopontin expressed in host cells. Moreover, such an antibody can be used to detect osteopontin (e.g., in a cellular lysate or cell supernatant) to evaluate the abundance and pattern of expression of osteopontin. Osteopontin antibodies can be used diagnostically to monitor protein levels in tissue as part of a clinical testing procedure, for example, to determine the efficacy of a given treatment regimen.

Antibodies found to activate the osteopontin receptor may be modified to minimize activities extraneous to osteopontin-mediated functions, such as IL12 production. Thus, the antibody molecules can be truncated or mutated to minimize or to remove activities outside of osteopontin activation. For example, for certain antibodies, only the antigen-binding portion is needed. Thus, the F_{c} portion of the antibody can be removed.

Cells expressing the osteopontin receptor are exposed to antibody for activation. Activated cells then are exposed to various molecules with a view to identifying those that alter receptor activity, whether to higher activation levels or to lower activation levels. Molecules that achieve those goals then can be tested on cells expressing the osteopontin receptor without antibody to observe the effect on non-activated cells. The target molecules then can be tested and modified as candidate drugs for the treatment of disorders associated with altered osteopontin metabolism using known techniques.

The screening assays of the instant invention are amenable to use with both the soluble form and membrane-bound osteopontin receptor. In the case of cell-free assays comprising the soluble osteopontin receptor, a membrane-solubilizing agent as known in the art is used to maintain the receptor in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton X-100, Triton X-114, Thesit®, isotridecylpoly(ethylene glycol ether)ₙ, 3-[(3-cholamidopropyl)dimethylammino]-1-propane sulfonate (CHAPS), 3-[(3-cholamidopropyl)dimethylammino]-2-hydroxy-1-propane sulfonate (CHAPSO) or N-dodecyl=N,N-dimethyl-3-ammonio-1-propane sulfonate.

It may be desirable to immobilize osteopontin, the receptor thereof or a target molecule thereof to facilitate separation of complexed from uncomplexed forms of one or both of the proteins, as well as to accommodate automation of an assay. Binding of a test compound to osteopontin or the receptor thereof, or interaction of either molecule with a target molecule in the presence and absence of a candidate compound, can be accomplished in any vessel suitable for containing the reactants. Examples of such vessels include microtitre plates, test tubes and micro-centrifuge tubes. In one embodiment, a fusion protein can be provided that adds a domain that allows one or both of the proteins to be bound to a matrix. For example, glutathione-S-transferase/osteopontin fusion proteins or glutathione-S-transferase/target fusion proteins can be adsorbed onto glutathione Sepharose® beads (Sigma Chemical, St. Louis, Mo.). Alternatively, glutathione-derivatized microtitre plates that then are combined with the test compound or the test compound and either the non-adsorbed target protein or osteopontin and the mixture incubated under conditions conducive to complex formation (e.g., at physiological conditions for salt and pH), can be used. Following incubation, the beads or microtitre plate wells are washed to remove any unbound components and complex formation is measured either directly or indirectly, for example, as described above. Alternatively, the complexes can be dissociated from the matrix and the level of osteopontin binding or activity determined using standard techniques.

Other techniques for immobilizing proteins on matrices also can be used in the screening assays of the invention. For example, osteopontin, the receptor thereof or a target molecule thereof can be immobilized utilizing conjugation of biotin and streptavidin. Biotinylated molecules can be prepared from biotin-NHS (N-hydroxy-succinimide) using techniques well known in the art (e.g., biotinylation kit, Pierce Chemicals, Rockford, Ill.) and immobilized in the wells of streptavidin-coated 96-well plates (Pierce Chemicals). Alternatively, antibodies reactive with osteopontin, the receptor thereof or target molecules but that do not interfere with binding of a target molecule can be derivatized to the wells of the plate and unbound target or osteopontin trapped in the wells by antibody conjugation. Methods for detecting such complexes, in addition to those described above for the GST-immobilized complexes, include immunodetection of complexes using antibodies reactive with osteopontin or target molecule, as well as enzyme-linked assays that rely on detecting an enzymatic activity associated with osteopontin.

In another embodiment, modulators of osteopontin function are identified in a method in which a cell is contacted with a candidate compound and the expression of induced mRNA or protein in the cell is determined. The level of particular mRNA or protein in the presence of the candidate compound is compared to the level of expression of MRNA or protein in the absence of the candidate compound. The candidate compound then can be identified as a modulator of osteopontin function based on that comparison.

In yet another aspect of the invention, osteopontin or the receptor thereof can be used as “bait proteins” in a two-hybrid assay or three-hybrid assay (see, e.g., U.S. Pat. No. 5,283,317; Zervos et al., Cell (1993) 72:223-232; Madura et al., J. Biol. Chem. (1993) 268:12046-12054; Bartel et al., Bio/Techniques (1993) 14:920-924; Iwabuchi et al., Oncogene (1993) 8:1693-1696; and PCT Publication No. WO 94/10300), to identify other proteins, that bind to or interact with osteopontin or the receptor thereof, and modulate osteopontin activity.

In another embodiment, it is desirable to minimize osteopontin production or reactivity, for example, when lack of oligodendrocyte maturation is desired. An antagonist of osteopontin can inhibit one or more of the activities of the naturally occurring osteopontin by, for example, competitively binding to the osteopontin receptor without activating same. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring osteopontin.

Antagonists of osteopontin can be identified as for agonists, as described hereinabove, for example, by screening combinatorial libraries of mutants, e.g., truncation mutants, or obtaining antagonistic antibodies.

As large quantities of pure osteopontin or receptor thereof can be made, physical characterization of the conformation of areas of likely function can be ascertained for rational drug design. Once the shape and ionic configuration of a region is discerned, candidate drugs that should interact with those regions can be configured and then tested in intact cells, animals and patients. Methods that would enable deriving such 3-D structure information include X-ray crystallography, NMR spectroscopy, molecular modeling and so on. The 3-D structure also can lead to identification of analogous conformational sites in other known proteins where known drugs that act at site exist.

As an agent that prevents differentiation of oligodendrocyte precursor cells and promotes proliferation of those precursor cells to increase the population thereof, osteopontin can be used therapeutically as discussed hereinabove to promote the growth and expansion of an oligodendrocyte population at a particular site in the body. Because oligodendrocytes produce myelin in the central nervous system, the use of osteopontin is a means for encouraging remyelination at sites of demyelination in the central nervous system, for example, in cases of multiple sclerosis and other disorders characterized by demyelination.

Osteopontin binds to responsive cells that express receptor. Integrin receptors, such as αᵥβ₃, bind osteopontin. Osteopontin receptors are expressed at high levels in oligodendrocyte precursors.

Osteopontin stimulates IL12 production in cells expressing the αᵥ chain. The αᵥ chain is expressed highly in osteocytes, oligodendrocytes and glial cells, and in lysolecithin-treated animals. The αᵥ chain can be associated with a variety of β chains.

The α₆ chain is expressed well by astrocytes and microglial cells. β₁ and β₃ also are expressed well by those cell types. B₅, while expressed by astrocytes and microglial, is expressed at higher levels by oligodendrocytes and oligodendrocyte precursors.

The β₃ chain also is associated with IL12 expression. β₃ chain expression wanes with oligodendrocyte differentiation. The β₁ and β₅ chains are associated also with osteopontin metabolism.

Hence, another method for reducing the impact of osteopontin is to minimize expression of the receptors of osteopontin or to occupy the osteopontin receptors so that the receptors cannot engage osteopontin. That goal can be achieved by reducing expression of, for example, the αᵥ chain, β₃ chain or both. Reducing expression can be achieved for example, by site directed mutagenesis, using antisense molecules and so on. The gene sequences of αᵥ and β₃ are known and available to the artisan. For the purposes of the instant invention, inhibiting is considered synonymous with inactivating and other such terms, the effect being the osteopontin receptor does not operate at the same level without the intervention, and thus the functions associated with osteopontin, such as chemoattraction or preventing differentiation of precursors, are diminished.

Alternatively, a molecule that binds to the osteopontin receptor but without osteopontin activity can be used to block the receptor. Such molecules can be identified for example, by using cells expressing the osteopontin receptor. Those cells can be exposed to a number of molecules to identify those that bind to the receptor but do not activate the receptor. Methods for screening molecules for, for example, αᵥβ₃ binding activity, are known in the art, for example, using cells that express or overexpress αᵥβ₃ or cells transformed to express recombinant αᵥβ₃.

The receptor-inhibiting molecule can be any molecule, a peptide, carbohydrate, organic molecule or combination thereof. For example, many current drugs are unrelated to a particular receptor ligand but bear a conformation, charge or both that mimic that of the natural ligand, such as, osteopontin.

Methods for determining activation of the osteopontin receptor are known, such as production of IL12. Methods for assaying for IL12 are known in the art.

Yet another method for preventing binding of osteopontin to the receptor is to use antibody that binds to osteopontin or to the receptor in a manner that prevents osteopontin from binding to the receptor thereof. Methods for obtaining antibodies to osteopontin or the osteopontin receptor are known in the art. Of those that are formed to bind to osteopontin or the receptor thereof, a competitive array can be used to identify those antibodies that inhibit binding of osteopontin to the receptor thereof.

In another embodiment, a truncated or modified form of osteopontin that engages receptor but does not activate the receptor can be made as taught herein.

Because osteopontin acts via a cell surface receptor, the receptor triggers a cascade of molecular interactions between and among cytoplasmic and nuclear proteins and nucleic acids culminating in the activation and/or inactivation of particular genes, such as that for IL12. Those intracellular elements downstream from the receptor are suitable targets for enhancing or suppressing osteopontin function. A candidate compound found to intervene in the osteopontin signaling cascade may be a suitable drug target.

The nucleic acid molecules, osteopontin, the receptor thereof, antibodies and modulators thereof, such as agonists and antagonists of the invention can be incorporated into pharmaceutical compositions suitable for administration. Such compositions typically comprise the active agent and a pharmaceutically acceptable carrier. As used herein, the language “pharmaceutically acceptable carrier” is intended to include any and all solvents, dispersion media, excipients, carriers, diluents, coatings, antibacterial and antifungal agents, isotonic and absorption delaying agents and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds also can be incorporated into the compositions.

A pharmaceutical composition of the invention is formulated to be compatible with the intended route of administration. Examples of routes of administration include parenteral, e.g., intravenous, intradermal and subcutaneous, oral (e.g., inhalation), transdermal (topical), transmucosal and rectal administration. Solutions or suspensions used for parenteral, intradermal or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose. The pH can be adjusted with acids or bases, such as HCl or NaOH. The parenteral preparation can be enclosed in ampoules, disposable syringes or multiple dose vials made of glass or plastic for storage.

Pharmaceutical compositions suitable for injectable use include sterile aqueous solutions (water miscible) or dispersions and sterile powders for the extemporaneous preparation of sterile injectable solutions or dispersions. For intravenous administration, suitable carriers include physiological saline, bacteriostatic water, Cremophor EL® (BASF; Parsippany, N.J.) or phosphate buffered saline (PBS). In all cases, the composition must be sterile and should be fluid to the extent that easy syringability exists. The composition must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyetheylene glycol and the like) and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols such as mannitol, sorbitol or sodium chloride, in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of the active ingredient plus any additional desired ingredient from a previously sterile-filtered solution thereof.

Oral compositions generally include an inert diluent or an edible carrier. The compositions can be enclosed in gelatin capsules or compressed into tablets. For the purpose of oral therapeutic administration, the active compound can be incorporated with excipients and used in the form of tablets, troches or capsules. Oral compositions also can be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients or compounds of a similar nature: a binder such as microcrystalline cellulose, gum tragacanth or gelatin; an excipient such as starch or lactose, a disintegrating agent such as alginic acid, Primogel or corn starch; a lubricant such as magnesium stearate or Sterotes; a glidant such as colloidal silicon dioxide; a sweetening agent such as sucrose or saccharin; or a flavoring agent such as peppermint, methyl salicylate or orange flavoring. For administration by inhalation, the compounds are delivered in the form of an aerosol spray from a pressurized container or dispenser that contains a suitable propellant, e.g., a gas such as carbon dioxide or a nebulizer.

Systemic administration also can be by transmucosal or transdermal means. For transmucosal or transdermal administration, penetrants appropriate to the barrier to be permeated are used in the formulation. Such penetrants generally are known in the art and include, for example, for transmucosal administration, detergents, bile salts and fusidic acid derivatives. Transmucosal administration can be accomplished through the use of nasal sprays or suppositories. For transdermal administration, the active compounds are formulated into ointments, salves, gels or creams as generally known in the art.

The compounds also can be prepared in the form of suppositories (e.g., with conventional suppository bases with lubricating materials of melting temperatures at mammalian body temperature, such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid.

Methods for preparation of such formulations will be apparent to those skilled in the art. The materials also can be obtained commercially from Alza Corporation and Nova Pharmaceuticals, Inc. Liposomal suspensions (including liposomes targeted to infected cells with monoclonal antibodies) also can be used as pharmaceutically acceptable carriers. Those can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. Depending on the type and severity of the disease, about 1 μg/kg to 15 mg/kg (e.g., 0.1 to 20 mg/kg) of antibody is an initial candidate dosage for administration to the patient, whether, for example, by one or more separate administrations or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of the therapy is monitored easily by conventional techniques and assays. An exemplary dosing regimen is disclosed in WO 94/04188. The specification for the dosage unit forms of the invention are dictated by and directly dependent on the unique characteristics of the active compound and the particular therapeutic effect to be achieved and the limitations inherent in the art of compounding such an active compound for the treatment of individuals.

Because the osteopontin can find use with demyelination disorders, the active agent may be preferably delivered to the central nervous system. Thus, the active agent can be infused into spinal fluid or directly into the brain using known stereotactic methods. Methods for enabling drugs to traverse the blood-brain barrier are known.

All references cited herein are herein incorporated in entirety.

It will be evident to those with skill in the art that various modifications and changes can be made to the teachings of the instant specification without departing from the spirit and scope of the invention.

We claim:

1. A method of modulating differentiation of oligodendrocytes comprising reducing exposure of oligodendrocytes and precursors thereof to osteopontin.

2. The method of claim 1, wherein said reducing is obtained by exposing said oligodendrocytes and precursors thereof to an antibody that specifically binds osteopontin.

3. The method of claim 1, wherein said reducing is obtained by inactivating osteopontin receptor.

4. The method of claim 3, wherein said receptor is exposed to an osteopontin antagonist.

5. The method of claim 3, wherein said receptor is exposed to an antibody that binds to said receptor.

6. A method of modulating differentiation of oligodendrocytes comprising modulating the activity of a receptor for osteopontin on oligodendrocytes or precursors thereof.

7. A method of inducing remyelination at a site requiring remyelination comprising reducing exposure of oligodendrocyte and precursor cells thereof at said site to osteopontin to enhance oligodendrocyte precursor number at said site, and then increasing exposure of said precursor cells to osteopontin to enhance differentiation into oligodendrocytes, wherein said oligodendrocytes enhance remyelination.

8. The method of claim 7, wherein said reducing is obtained by using an antibody that specifically binds osteopontin.

9. The method of claim 7, wherein said reducing is obtained by inactivating osteopontin receptor.

10. The method of claim 7, further comprising exposing cells remote from said site to osteopontin, wherein osteopontin is a chemoattractant and causes migration of responsive cells to said site.

11. The method of claim 10, wherein said osteopontin is secreted by astrocytes.

12. The method of claim 10, wherein said osteopontin is expressed by cells exposed to an osteopontin agonist.

13. The method of claim 10, wherein said osteopontin is expressed by cells exposed to an antibody that binds to osteopontin receptor.

14. A method of obtaining a molecule that induces migration of cells to a site requiring myelination comprising:
exposing cells expressing an osteopontin receptor to candidate molecules;
identifying those candidate molecules that bind to said receptor;
exposing oligodendrocyte precursor cells to said identified candidate molecules; and
identifying those candidate molecules that induce migration of said precursor cells.

15. The method of claim 14, wherein said molecule that induces migration is an osteopontin agonist or inverse agonist.

16. A method of obtaining a molecule that induces dedifferentiation of an oligodendrocyte or prevents differentiation of an oligodendrocyte precursor cell comprising:
exposing cells expressing an osteopontin receptor to candidate molecules;
identifying those candidate molecules that bind to said receptor;
exposing oligodendrocyte precursor cells to said identified candidate molecules; and
identifying those candidate molecules that prevent differentiation of said precursor cells into mature oligodendrocytes.

17. A method of obtaining a molecule that induces dedifferentiation of an oligodendrocyte or prevents differentiation of an oligodendrocyte precursor cell comprising:
exposing cells expressing an osteopontin receptor to candidate molecules;
identifying those candidate molecules that bind to said receptor;
exposing oligodendrocytes to said identified candidate molecules; and
identifying those candidate molecules that induce dedifferentiation of said oligodendrocytes.
